SEQUENCE LISTING (29 sequences)

SEQ ID NO: 1 - DNA, 58 bp, Artificial
  Primer for echovirus 1 VP1 protein
aattattcgaattagcaagaagatattgtaccgaaattaatacgactcactatagggg

SEQ ID NO: 2 - DNA, 69 bp, Artificial
  Primer for echovirus 1 VP1 protein
taaataaagcggccgcttattactagtctttatctcctttgattgtaaataaaatgtaatttacagtat

SEQ ID NO: 3 - DNA, 42 bp, Artificial
  Primer for echovirus 1 VP3 protein
atagcatggtaccaccgaaattaatacgactcactatagggg

SEQ ID NO: 4 - DNA, 70 bp, Artificial
  Primer for echovirus 1 VP3 protein
aatactagttcgaaggtaggtagctagcgtatatctcctttgattgtaaataaaatgtaatttacagtat

SEQ ID NO: 5 - DNA, 43 bp, Artificial
  Primer for echovirus 1 VP2 protein
atattaggcgcgccaccgaaattaatacgactcactatagggg

SEQ ID NO: 6 - DNA, 67 bp, Artificial
  Primer for echovirus 1 VP2 protein
attaatctgcagatttataggcgccgtatatctcctttgattgtaaataaaatgtaatttacagtat

SEQ ID NO: 7 - DNA, 50 bp, Artificial
  Vectorial cloning primer for echovirus 1 VP1
gcttcactagttctgactgctaagcatgggtgatgtgcagaatgctgtcg

SEQ ID NO: 8 - DNA, 38 bp, Artificial
  Vectorial cloning primer for echovirus 1 VP1
caaggttgcggccgcgatgatcgttgttattatgttgg

SEQ ID NO: 9 - DNA, 41 bp, Artificial
  Vectorial cloning primer for echovirus 1 VP3
gcttcgctagcatgggactaccgaccatgaacacccctggc

SEQ ID NO: 10 - DNA, 39 bp, Artificial
  Vectorial cloning primer for echovirus 1 VP3
cggccattcgaactactattggtaaaaagatgtttgctc

SEQ ID NO: 11 - DNA, 35 bp, Artificial
  Vectorial cloning primer for echovirus 1 VP2
gcttcggcgccatgtctccaacggttgaagagtgc

SEQ ID NO: 12 - DNA, 37 bp, Artificial
  Vectorial cloning primer for echovirus 1 VP2
gccggaactgcagctactattggtgtccagctagtcg

SEQ ID NO: 13 - DNA, 40 bp, Artificial
  Vectorial cloning primer for echovirus 1 VP4
gcttcagatatcggtatcaacacagaagaccggggcgcac

SEQ ID NO: 14 - DNA, 96 bp, Artificial
  Vectorial cloning primer for echovirus 1 VP4
gccatccggagatctctactacgaattcagcgctggcagggtttttatcatgacatctttcattggttcagtaaacttcccggggtcttgggtgaa

SEQ ID NO: 15 - DNA, 30 bp, Artificial
  Vectorial cloning primer containing spe1 restriction site
gcgcgactagtctgatggactgcgacatcg

SEQ ID NO: 16 - DNA, 35 bp, Artificial
  Vectorial cloning primer containing bpu1102I restriction site
cacccatgcttagcgcgtgttctttagtgcccatc

SEQ ID NO: 17 - DNA, 27 bp, Artificial
  Vectorial cloning primer containing avri restriction site
gctcgcccgggatggactgcgacatcg

SEQ ID NO: 18 - DNA, 34 bp, Artificial
  Vectorial cloning primer containing bgl ii restriction site
gccatccggagatctctagtgttctttagtgccc

SEQ ID NO: 19 - DNA, 28 bp, Artificial
  Vectorial cloning primer containing eco ri restriction site
gcgctgaattcgatggactgcgacatcg

SEQ ID NO: 20 - DNA, 945 bp, Artificial
  Recombinant echovirus 1 VP1 shell protein
atgggtgatgtgcagaatgttgtcgaaggggctatggtcagggtggcagatacagtgcaaacttcagccacaaactcagagagggtgcctaacttgacagcagtagaaactggtcacacttcgcaggtagtacctggtgataccatgcagaccagacatgtgatcaacaatcacgtgaggtcagaatctacaattgagaacttccttgccagatcagcgtgtgttttcttgctagagtacaagacagggaccaaagaggattccaatagcttgaacaatggggtgattacaaccaggcgagtgggtcaactacgtataaaactggaaatgtttacttacctacggtttgacatggaaatcaccgtggtcattacaagctcgcaagatcagtctacatcacaaaaccagaatgcaccagtgctaacacaccagataatgtatgtaccaccagggggacccatacccataagcgtggatgattacagctggcaaacattcaccaaccccagtatcttttggaccgaagggaacgctccggcacgcatgtcaattccatttattaacataggcaatgcgtatagtaatttctacgatgggtggtctcacttctcccaggctggcgtgtatggcttcactactctgaacaacatgggtcaattgttcttccggcacgtaaacaagcccaacccagccgctattacaagtgtggcgcgcatttacttcaaaccgaaacatgtacgcgcttgggtgcctagaccaccgcgcttgtgtccatacatcaatagcacgaatgtcaactttgaacccaagccagtgactgaagtacgtaccaacataataacaacgatcatcgcggccgcacagctgtatacacgtgcaagccagccagaactcgccccggaagaccccgaggatctcgagcaccaccatcaccatcaccatcac

SEQ ID NO: 21 - DNA, 843 bp, Echovirus 1
  gene (1)..(843): Native VP1 shell protein
ggtgatgtgcagaatgctgtcgaaggggctatggtcagggtggcagatacagtgcaaacttcagccacaaactcagagagggtgcctaacttgacagcagtagaaactggtcacacttcgcaggcagtacctggtgataccatgcagactagacatgtgatcaacaatcacgtgaggtcagaatctacaattgagaacttccttgccagatcagcgtgtgttttctacctagagtacaagacagggaccaaagaggattccaatagcttcaacaattgggtgattacaaccaggcgagtggctcaactacgtagaaaactggaaatgtttacttacctacggtttgacatggaaatcaccgtggtcattacaagctcgcaagatcagtctacatcacaaaaccagaatgcaccagtgctaacacaccagataatgtatgtaccaccagggggacccatacccgtaagcgtggatgattacagctggcaaacatccaccaaccccagtatcttttggaccgaagggaacgctccggcacgcatgtcaattccatttattagcataggcaatgcgtatagtaatttctacgatgggtggtctcacttctcccaggctggcgtgtatggcttcactactctgaacaacatgggtcaattgttcttccggcacgtaaacaagcccaacccagccgctattacaagtgtggcgcgcatttacttcaaaccgaaacatgtacgcgcttgggtgcctagaccaccgcgcttgtgtccatacatcaatagcacgaatgtcaactttgaacccaagccagtgactgaagtacgtaccaacataataacaacg

SEQ ID NO: 22 - DNA, 792 bp, Artificial
  Recombinant echovirus 1 VP2 shell protein
atgtctccaacggttgaagagtgcgggtacagtgacagggtcaggtcaatcacacttgggaactccactattacaactcaagagtgtgccaatgtggtggtggggtacggtgaatggcctgagtatctgagtgataacgaggcaactgctgaggaccaaccaacgcagccggacgtggccacttgccgtttttacaccctagactcagtccaatgggagaatgggtcaccaggttggtggtggaagtttcccgacgctctaagggatatgggattatttggccaaaatatgtactaccattacttaggcagagccgggtataccatccacgtacaatgcaatgcttccaagtttcatcaaggctgtatcctggtagtgtgtgtccctgaggcggagatgggaagtgcccaaacctcaggggtggtcaactacgaacacattagtaagggtgagatcgcatcaaggttcactaccacgacaacagcagaagaccatggcgtgcaggccgcggtatggaatgctggtatgggcgttggagttgggaacttgacgatcttcccgcaccaatggatcaaccttcgcaccaacaacagcgccacaattgttatgccatacgtaaatagtgtaccaatggacaatatgtatagacatcacaactttacactaatgataataccctttgtgcctctggatttcagcgcgggtgcatccacatacgtgcccataacggtgacagtggcccccatgtgtgccgagtacaatggactacgactagctggacaccaatagtag

SEQ ID NO: 23 - DNA, 783 bp, Echovirus 1
  gene (1)..(783): Native VP2 shell protein
tctccaacggttgaagagtgcgggtacagtgacagggtcaggtcaatcacacttgggaactccactattacaactcaagagtgtgccaatgtggtggtggggtacggtgaatggcctgagtatctgagtgataacgaggcaactgctgaggaccaaccaacgcagccggacgtggccacttgccgtttttacaccctagactcagtccaatgggagaatgggtcaccaggttggtggtggaagtttcccgacgctctaagggatatgggattatttggccaaaatatgtactaccattacttaggcagagccgggtataccatccacgtacaatgcaatgcttccaagtttcatcaaggctgtatcctggtagtgtgtgtccctgaggcggagatgggaagtgcccaaacctcaggggtggtcaactacgaacacattagtaagggtgagatcgcatcaaggttcactaccacgacaacagcagaagaccatggcgtgcaggccgcggtatggaatgctggtatgggcgttggagttgggaacttgacgatcttcccgcaccaatggatcaaccttcgcaccaacaacagcgccacaattgttatgccatacgtaaatagtgtaccaatggacaatatgtatagacatcacaactttacactaatgataataccctttgtgcctctggatttcagcgcgggtgcatccacatacgtgcccataacggtgacagtggcccccatgtgtgccgagtacaatggactacgactagctggacaccaa

SEQ ID NO: 24 - DNA, 937 bp, Artificial
  Recombinant echovirus 1 VP3 shell protein
atgtgcagaatgttgtcgaaggggctatggtcagggtggcagatacagtgcaaacttcagccacaaactcagagaggtgcctaacttgacagcagtagaaactggtcacacttcgcaggtagtacctggtgataccatgcagaccagacatgtgatcaacaatcacgtgaggtcagaatctacaattgagaacttccttgccagatcagcgtgtgttttcttgctagagtacaagacagggaccaaagaggattccaatagcttgaacaatggggtgattacaaccaggcgagtgggtcaactacgtataaaactggaaatgtttacttacctacggtttgacatggaaatcaccgtggtcattacaagctcgcaagatcagtctacatcacaaaaccagaatgcaccagtgctaacacaccagataatgtatgtaccaccagggggacccatacccataagcgtggatgattacagctggcaaacattcaccaaccccagtatcttttggaccgaagggaacgctccggcacgcatgtcaattccatttattaacataggcaatgcgtatagtaatttctacgatgggtggtctcacttctcccaggctggcgtgtatggcttcactactctgaacaacatgggtcaattgttcttccggcacgtaaacaagcccaacccagccgctattacaagtgtggcgcgcatttacttcaaaccgaaacatgtacgcgcttgggtgcctagaccaccgcgcttgtgtccatacatcaatagcacgaatgtcaactttgaacccaagccagtgactgaagtacgtaccaacataataacaacgatcatcgcggccgcacagctgtatacacgtgcaagccagccagaactcgccccggaagaccccgaggatctcgagcaccaccatcaccatcaccatcac

SEQ ID NO: 25 - DNA, 843 bp, Echovirus 1; identical to SEQ ID NO: 21
  gene (1)..(843): Native VP3 shell protein

SEQ ID NO: 26 - DNA, 213 bp, Artificial
  Recombinant echovirus 1 VP4 shell protein
atggcgatatcggtatcaacacagaagaccggggcgcacgagactagcttgagcgctactggcaactccataatacactacacgaacattaattattacaaagatgcagcctctaactctgccaatagacaagatttcacccaagaccccgggaagtttactgaaccaatgaaagatgtcatgataaaaaccctgccagcgctgaattcgtag

SEQ ID NO: 27 - DNA, 207 bp, Echovirus 1
  gene (1)..(207): Native VP4 shell protein
atgggagcacaggtatcaacacagaagaccggggcgcacgagactagcttgagcgctactggcaactccataatacactacacgaatattaattattacaaagatgcagcctctaactctgccaatagacaagatttcacccaagaccctggtaagtttactgaaccaatgaaagatgtcatgataaaaaccctgccagcgctgaat

SEQ ID NO: 28 - DNA, 1047 bp, Homo sapiens
  ref <301>: AUTHORS: Hatada,S., Seed,J.R., Barker,C., Hajduk,S.L., Black,S. and Maeda,N.
  ref <302>: TITLE: No trypanosome lytic activity in the sera of mice producing human
  ref <303>: JOURNAL: Mol. Biochem. Parasitol.
  ref <304>: VOLUME: 119
  ref <305>: ISSUE: 2
  ref <306>: PAGES: 291-294
  ref <307>: DATE: 2002-02-01
  ref <313>: RELEVANT RESIDUES: (1)..(1047)
atgagtgacctgggagctgtcatttccctcctgctctggggacgacagctttttgcactgtactcaggcaatgatgtcacggatatttcagatgaccgcttcccgaagccccctgagattgcaaatggctatgtggagcacttgtttcgctaccagtgtaagaactactacagactgcgcacagaaggagatggagtatacaccttaaatgataagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggggccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggtggttctacaccctaactaccaccaggtagatattgggctcatcaaactcaaacagaaggtgcttgttaatgagagagtgatgcccatctgcctaccttcaaagaattatgcagaagtagggcgtgtgggttacgtgtctggctggggacaaagtgacaactttaaacttactgaccatctgaagtatgtcatgctgcctgtggctgaccaatacgattgcataacgcattatgaaggcagcacatgccccaaatggaaggcaccgaagagccctgtaggggtgcagcccatactgaacgaacacaccttctgtgtcggcatgtctaagtaccaggaagacacctgctatggcgatgcgggcagtgcctttgccgttcacgacctggaggaggacacctggtacgcggctgggatcctaagctttgataagagctgtgctgtggctgagtatggtgtgtatgtgaaggtgacttccatccaggactgggttcagaagaccatagctgagaactaa

SEQ ID NO: 29 - protein, 348 aa, Homo sapiens
  ref <301>: AUTHORS: Hatada,S., Seed,J.R., Barker,C., Hajduk,S.L., Black,S. and Maeda,N.
  ref <302>: TITLE: No trypanosome lytic activity in the sera of mice producing human
  ref <303>: JOURNAL: Mol. Biochem. Parasitol.
  ref <304>: VOLUME: 119
  ref <305>: ISSUE: 2
  ref <306>: PAGES: 291-294
  ref <307>: DATE: 2002-02-01
  ref <313>: RELEVANT RESIDUES: (1)..(348)
MSDLGAVISLLLWGRQLFALYSGNDVTDISDDRFPKPPEIANGYVEHLFRYQCKNYYRLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYHQVDIGLIKLKQKVLVNERVMPICLPSKNYAEVGRVGYVSGWGQSDNFKLTDHLKYVMLPVADQYDCITHYEGSTCPKWKAPKSPVGVQPILNEHTFCVGMSKYQEDTCYGDAGSAFAVHDLEEDTWYAAGILSFDKSCAVAEYGVYVKVTSIQDWVQKTIAEN

(19) United States
(12) Patent Application Publication
(10) Pub. No.: US 2005/0214264 A1
(43) Pub. Date: Sep. 29, 2005

(54) Sacromastigophoric therapeutic agent delivery system

(76) Inventor: Lawrence W. Cosenza, Birmingham, AL (US)

Correspondence Address: GIFFORD, KRASS, GROH, SPRINKLE & CITKOWSKI, P.C, PO BOX 7021, TROY, MI, 48007-7021, US

(21) Appl. No.: 10/735,203

(22) Filed: Dec. 12, 2003

Related U.S. Application Data
(60) Provisional application No. 60/433,269, filed on Dec. 13, 2002.

(51) Int. Cl. A61K 48/00; C12N 1/10

(52) U.S. Cl. 424/93.21; 435/258.1

(57) ABSTRACT

A sacromastigophoric organism such as a trypanosome is harnessed for its ability to evade a host immune system, as well as its vast carrying capability to deliver a therapeutic agent to a host. The organism is engineered to include a recombinant lytic factor. After administration of the live organism to a host, an exogenous species is administered to induce organism lysis thereby releasing the organism contents into the host environment. The organism readily delivers genes and non-nucleic acid therapeutic agents to a host.

### RELATED APPLICATION

This application claims priority of U.S. Provisional Patent Application Ser. No. 60/433,269 filed Dec. 13, 2002, which is incorporated herein by reference.

### BACKGROUND OF THE INVENTION

Viral and artificial membrane based therapeutics have failed in clinical environments for two primary reasons, antigenicity and specificity (Damian 1997). Gene therapy is defined by the introduction of foreign genes into a host genome with the anticipation of correction of genetic defect, development of a desired phenotype, or vaccination against a tumor or foreign microorganism. The attraction of the ability to correct genetic deficiencies with a single therapy has promoted tremendous activity in gene therapy research. Currently there are two approaches to delivering genetic information to a cell, one uses an engineered viral system and the other encapsulates DNA with artificial membranes. Both of these systems are antigenic and display non-specific targeting (Lotze and Kost 2002). The antigenicity derived from the therapy is self-defeating because the host immune system clears the active agent from the bloodstream prior to therapeutic activity. Hence, the gene delivery vector is removed from the host before integration can take place. Provided the gene delivery vector can avoid eliciting an immune response, it is important to have cell or tissue specific targeting. It is important to deliver the gene and ultimately the expressed gene product to the area or place where the desired activity is required in order to address a disease state. For example, sickle cell anemia is a disease of the blood and delivery of a gene designed for its treatment to the lung may not have clinical significance. Additionally, delivery of a gene to unintended tissues may have unanticipated toxicities. Therefore current therapy research has focused on developing products and methods for reducing antigenicity and increasing target specificity for gene delivery.

Technical constraints limit the scope of gene therapies. Physical limitations in the size and number of genes that a viral system can carry reduce the number of applications available for a gene therapy approach. Low rates of target gene integration are related to rates of infection as well as the efficiency of integration. The retroviral vectors are the vehicles of choice for large gene delivery systems. Currently, the retroviral delivery systems can accommodate gene target inserts up to 7 kb. If retroviral systems were clinically effective, they would only have efficacies for monocistic genetic disorders and would be of little use for diseases of polycistronic nature (Weber and Fussenegger 2002). This limitation may accommodate the transfer of single genes but becomes obsolete for the correction of multi-gene systems.

Representative of the sacromastigophoric protozoa that are able to evade immune systems is Trypanosoma brucei. Trypanosoma brucei is a pathological organism that has the ability to evade human immune system surveillance, specifically infect cells that display a receptor for gp83 trans-sialidase (gp83-TSA) expressed on the surface of trypanosome and a natural ability to transfer genetic information to its host. Trypanosome brucei evades the immune system by continually changing its surface coat, can live in the bloodstream for an indefinite period of time, and has an unlimited capacity for carrying genetic material. These characteristics are attractive for developing the trypanosome organism into an allogous cell-based protein delivery system. Trypanosomatids have a digenetic life cycle that involves mammalian and insect hosts and are responsible for a variety of diseases in humans and domestics animals. The trypanosoma genus contains three human and a number of animal pathogens. The Central and South American Trypanosoma cruzi is an intercellular parasite responsible for Chagas' disease. Trypanosoma brucei subspecies rhodesiense and gambiense are the causative agents of sleeping sickness. Trypanosoma brucei brucei causes Nagana, a wasting disease in domestic animals. The T. brucei brucei strain, which is not pathogenic for humans, can live in the bloodstream. While in the mammalian bloodstream T. brucei brucei is protected from the immune system by a variable surface glycoprotein (VSG) coat. This organism is morphologically and biochemically similar to the other two T. brucei subspecies but is sensitive to a non-immune factor in human serum called haptoglobin-related protein (Hrp).

These disease states and the difficulty in treating patents with this infection are related to the parasite's ability to avoid immune system surveillance (Damian 1997). In fact, humans do not develop immunity to trypanosome after infection and are subject to re-infection. Trypanosoma infect myoblast, fibroblast and macrophages through interaction with gp83-TSA (Villalta et al. 2001).

Cell lysis and or programmed cell death can be triggered in trypanosomes by the addition of various molecules. For nearly a century it has been known that normal human serum can lyse Trypanosoma brucei brucei. This phenomenon is caused by a non-immune factor, trypanosome lytic factor (TLF) (Bishop et al., 2001; Hajduk and Moore, 1989). TLF exists in two forms. TLF-1 is a subspecies of high-density lipoprotein and TLF-2 is a large multimer that contains IgM and other proteins. Haptoglobin-related protein is a factor common to both types of TLF (Shimamura et al., 2001). Trypanosome cell lysis is induced after Hpr is incorporated into the lysosome subsequent to receptor uptake in the flagellar pocket. The mechanism of cell lysis is not well understood. At least two models have been proposed. In both Hpr is the toxin that is internally localized to and subsequently causes degradation of the lysosomal membranes. Release of proteolytic enzymes is then proposed to initiate demise of the organism.

The trypanosome organism carries small genetic elements that can be transferred between trypanosome parasites and the host of infection (Teixeira et al. 1994; Turner et al. 1990). These transducible elements (Lai 1994) have been characterized as mini-circles in one publication. The trypanosomes are single celled flagellated protozoan organisms that can accept plasmid DNA like a bacterium. Interestingly, plasmids that act as inducible expression vectors have been developed and commercially marketed for the expression of foreign genes in trypanosome (Furger et al. 2001). These plasmids utilize the T7 promoter commonly found in bacterial systems and can accommodate the inclusion of several genes including those used for initiating a novel form of programmed cell death and selection markers (Bishop et al. 2001; Shimamura et al. 2001; Stoka et al. 2001; Pearson et al. 2000). Thus, there exists a need for a therapeutic agent delivery system that has greater carrying capacity, target cell specificity, and avoids complications associated with immune system activation.

### SUMMARY OF THE INVENTION

A therapeutic delivery system for a host includes a therapeutic agent and a sacromastigophoric organism containing the therapeutic agent and a recombinant lytic factor, the lytic factor being upregulated by a promoter responsive to an induction species exogenous to both the organism and the host. In this way, the therapeutic agent is delivered by way of an organism capable of evading the host immune system and being subject to lysis upon administration of the induction species. In its simplest form, organism lysis confers post-immunity to the organism. Optionally, therapeutic agents such as nucleic acid sequences making up genes, artificial chromosomes and the like; magnetic species, radioactive species, vitamins; nanocrystals; drugs; and prodrugs function as the therapeutic agent alone, or in combination.

Sacromastigophoric organisms for delivery of a therapeutic agent are produced by a method including culturing such organisms that have been transfected with an expression cassette induced by an exogenous species, the cassette includes a first construct having a promoter controlling expression of a lytic protein. When the cassette also includes a ribosome binding site and a therapeutic gene, the therapeutic gene is capable of translation into a polypeptide. A polymerase termination sequence proximal to the therapeutic gene facilitates translation at the appropriate polypeptide length. A polypeptide expressed within a sacromastigophoric organism prior to lysis is readily glycosylated and otherwise modified by the organism machinery to yield a completed protein prior to organism lysis.

The treatment or prevention of a disease in a host is provided by administering to the host a therapeutic amount of sacromastigophoric organism that has been transfected with an expression cassette induced by an exogenous species signal where the cassette includes a first construct having a promoter controlling expression of a lytic protein. After allowing sufficient time for the organism to infect the host, the exogenous species is administered to induce lysis of the organism.

### BRIEF DESCRIPTION OF THE DRAWING

FIG. 1 is a schematic representation of a regulated tetracycline inducible expression construct operative in an inventive delivery system;

FIG. 2 shows amplification of VP1-4 capsid genes from the RNA genome of EV1 using RT-PCR;

FIG. 3 shows PCR duplication of T7lac and p10 promoters from pTriEx-1.1;

FIG. 4 shows a comparison of duplicated composite promoters;

FIG. 5 shows native and recombinant VP1 gene DNA sequence comparison;

FIG. 6 shows native and recombinant VP2 gene DNA sequence comparison;

FIG. 7 shows native and recombinant VP3 gene DNA sequence comparison;

FIG. 8 shows native and recombinant VP4 gene DNA sequence comparison;

FIG. 9 is a diagram of pLCRC, a multiple gene expression vector for use in trypanosome;

FIG. 10 is a diagram of a haptoglobin-related protein construct showing coding sequence as the N-terminal antibody tag and a bipartite C-terminal sequence for targeting to a lysosome; and

FIG. 11 is a DNA and amino acid sequence for haptoglobin related protein showing the codons, gene and single nucleotide polymorphisms.

### DETAILED DESCRIPTION OF THE PREFERRED EMBODIMENTS

The present invention has utility in delivering a therapeutic agent to a host where the therapeutic agent is a gene or a non-nucleic acid therapeutic agent. The present invention, in contrast to the prior art, avoids eliciting a host immune response and unlike conventional vectors has a near unlimited transport capability allowing for the first time the delivery to a host of components as large as artificial chromosomes, nanorobots, micromachines, nanocrystals and the like.

As used herein, a “gene” is defined to be an isolated nucleic acid molecule of greater than twenty nucleotides. A gene operative herein is recognized to be one that illustratively replaces or supplements a desired finction, or achieves a desired effect such as the inhibition of tumor growth or induction of an immune response to the gene itself or a polypeptide transcribed therefrom. The therapeutic proteins expressed through gene expression according to the present invention are virtually limitless. Proteins illustratively include insulin; interferons; insulin-like growth factors; glucocerebrosidase; toxins targeting cancer cells; immunogenic proteins, such as pathogenic viral protein; and antibiotic proteins, such as mutacins. It is appreciated that a nucleic acid molecule according to the present invention illustratively includes artificial chromosomes, plasmids, vectors, external guide sequences for RNAase, ribozymes, DNA, RNA, miRNA, and sRNA. Anti-sense nucleic acids sequences are also administered according to the present invention. A gene is generally under the control of an appropriate promoter, which may be inducible, repressible, or constitutive. Promoters can be general promoters, yielding expression in a variety of mammalian cells, or cell specific, or even nuclear versus cytoplasmic specific. Viral promoters such as CMV are also operative herein. These are known to those skilled in the art and can be constructed using standard molecular biology protocols.

RNAi mechanisms have now been found in a wide variety of cell types and shown to control expression of genes post-transcriptionally including those genes expressed as a result of viral infection, mutagens and cancers. mRNA degradation has been shown to be responsive to the presence of very short 21-23 base, double-strand, complementary RNA to preclude translation into functional proteins. S. M. Hammond et al. (2001); G. Hutvágner et al., Curr. Opin. Genet. Dev. 12, 225-232 (2002); P. A. Sharp et al. (2001); P. M. Waterhouse et al., (2001); G. Hutvágner et al., Science 297, 2056-2060 (2002). The process of RNAi is now known to involve Dicer enzyme that cleaves double-stranded RNA into small RNA fragments. These small RNA fragments are classified as either micro-RNA (miRNA) and small interfering RNA (siRNA) based on their ultimate function or mechanism of regulation. RNAi is brought about by the small RNA fragments degrading the mRNA in the case of siRNA or in the case of miRNA simple binding to the mRNA that codes for a protein sequence through the action of the ribosome. RISC enzyme complex has been implicated in assisting the binding of the small RNA fragments to identify complementary sequence and degrade mRNA. RNAi has also been implicated in modifying gene expression across generations without changes in cellular DNA sequences, commonly referred to as epigenetics. J. Couzin, Science 298, 2296-2297 (2002). The ability to silence an existing host gene or facilitate expression of a gene delivered according to the present invention is appreciated to be an aspect of the present invention operative for long-term genetic expression or suppression.

As used herein, a “host” includes humans, non-human primates, horses, goats, cows, sheep, pigs, dogs, cats, rodents, birds, fish, and plants. The methods and compounds of the present invention are administered in therapeutically effective amounts.

As used herein, a “therapeutically effective amount” is defined to include an amount necessary to delay the onset of, inhibit the progress of, relieve the symptoms of, or reverse a condition being treated; induce an immune response to the delivered gene or a polypeptide encoded thereby or regulate the expression of an existing cellular product. The therapeutically effective amount is one that is less than that that produces medically unacceptable side effects. It is appreciated that a therapeutically effective amount varies with a number of factors illustratively including subject age, condition, sex and the nature of the condition being treated. It is further appreciated that determining a therapeutically effective dose is within the knowledge of one of ordinary skill in the art.

The term “amino acid” is intended to include D- and L-form amino acids and modified or synthetic amino acids.

As used herein “non-nucleic acid therapeutic agent” is defined to be a magnetic species, radioactive species, a vitamin, a nanocrystal, a drug, and a prodrug. It is appreciated that a diagnostic marker that allows for identification and or quantification of cells or tissues that have a particular attribute such as cancerous growth, particular cellular receptors or the like constitutes a therapeutic function through diagnosis. Magnetic species and radioactive species are representative of diagnostic markers that also may be used as part of a direct thermal or oncological therapy, as is known to the art.

The term “drug” is defined as compounds used as a medicine to treat a disease, disease system, or undiagnosed pain. Drugs, as used herein, are typically small organic molecules having a molecular weight of less than 1000 and illustratively include channel blockers, receptor blockers, steroids, opioids, platinum compounds, terpenoids, and alkaloids. As used herein, drugs include pharmaceutically acceptable salts, esters, and amides of active medicinal species.

The term “prodrug” is defined as compounds that are rapidly transformed in vivo to yield the parent compounds of the above formula, for example, by hydrolysis in blood. (T. Higuchi et al. 1987).

The mode of administration according to the present invention is dictated by convenience and survival of at least a portion of the administered population of sacromastigophoric organisms. Preferably, an organism according to the present invention is delivered parentally. More preferably, the inventive organism is delivered by intravenous parenteral injection. It is appreciated that other routes of administration are also operative herein, these alternate routes illustratively including intracisternally, intrathecally, intravaginally, intraperitoneally, intravesically, or as a buccal or nasal spray.

Compositions suitable for parenteral injection optionally include physiologically acceptable sterile aqueous or non-aqueous solutions, dispersions, suspensions or emulsions. Examples of suitable aqueous and non-aqueous carriers, diluents, solvents and vehicles illustratively include water; ethanol; polyols, such as propylene glycol, polyethylene glycol, glycerol, and the like; combinations thereof; and injectable organic esters, such as ethyl oleate with the proviso that the carriers, diluents, solvents and vehicles are compatible with retaining the viability of the recombinant sacromastigophoric organism.

Inventive compositions optionally also include adjuvants such as preservatives, and organism nutritional factors. Prevention of the action of opportunistic microorganisms is assured through the addition of various antibacterial and antifungal agents, illustratively including parabens, chlorobutanol, phenol, sorbic acid, and the like with the proviso that the recombinant sacromastigophoric organism is not killed by the antibacterial and antifungal agents. Isotonic agents are also optionally operative herein and illustratively include sugars, sodium chloride and the like.

A recombinant sacromastigophoric organism according to the present invention includes a lytic factor activated by an exogenous species to both the organism and the host. An inventive sacromastigophoric organism that is able to evade a host immune response optionally is further genetically modified to accommodate many of the gene delivery vectors already developed, or serve as a targeting delivery system for delivering non-nucleic acid therapeutic agents to targeted host cells, naturally avoiding the human immune system. While the present invention is detailed herein with respect to trypanosomes, it is appreciated that the teachings of this prototypical system are indicative of other immune evading sacromastigophoric organisms are Amoeba, Giardia, Entamoeba, and Leishmania. Additionally, Plasmodia are operative herein. Trypanosomes display a natural tendency to transfer genetic material between themselves and a eukaryotic host. Research into the pathology of trypanosome has resulted in the development of inducible expression vectors for enhanced protein expression, anti-sense mRNA production and genetic engineering of the trypanosome organism (Santos and Buck 2000; Wirtz et al. 1999; Wen et al. 2001). These investigations have resulted in defining the function of specific molecules important for life cycle progression and infection. These vectors serve as a starting point for the development of an inventive trypanosome organism for delivering therapeutic agents to specific cell or tissue types in the absence of eliciting host immune system response and provide a trigger mechanism for the simultaneous release of engineered genes for homologous recombination and eliminating pathology due to the delivery vehicle (Trudeau et al. 2001). Additionally, a sacromastigophoric organism has the advantage of growing to greater density by about a factor of ten than mammalian cells and like other eukaryotic cells, modify proteins by glycosylation, tyrosine phosphorylation, and addition of glycolipid anchors. As any of these modifications may be crucial to full biological activity of expressed proteins, these organisms offer an advantage over bacterial systems. A comparison between the trypanosome and current retroviral gene delivery vectors is presented in Table 1.

TABLE 1
Comparison of inventive intracellular parasitic organism therapeutic
delivery system and prior art retroviral gene delivery system

| | Intracellular Parasitic | |
|---|---|---|
| | Organism Therapeutic | Prior Art Retroviral |
| | Delivery System | Gene Delivery Systems |
| Immune | Avoids immune system | Elicits immune system |
| system | activation through | activity. |
| activation | complement inactivation | |
| | and differential sur- | |
| | face protein expression. | |
| Tissue | Infects cells display- | Nonspecific. Specifi- |
| specificity | ing gp83-TSA receptor. | city can be altered |
| during | Infects myoblast, | using pseudotyping |
| infection | fibroblast and macro- | constructions. |
| | phages. | |
| Reversion to | None, there is no | Can revert to a rep- |
| replication | requirement for pack- | lication competent |
| competent | aging cells or plasmids | vector from a recom- |
| vector | with transgenes. | bination event be- |
| | | tween provirus vector |
| | | and the gag, -pol and |
| | | env plasmid trans- |
| | | genes. |
| DNA carrying | Not limited. May deliver | Limited to 7-8 kb. |
| capacity | multiple genes, whole | |
| | artificial chromosomes, | |
| | non-nucleic acid thera- | |
| | peutics. | |
| Titers | No foreseeable problems | Titers can be |
| | with generating large | problematic. |
| | enough titers for | |
| | therapeutic use. | |
| Controlled | The multiple gene ex- | Gene delivery is |
| release and | pression vector that | related to virus |
| integration of | drives the trypanosome | binding and infec- |
| provirus. | gene delivery system | tion. |
| | maybe engineered to | |
| | release provirus using | |
| | inducible promoters | |
| | like the T7p or Tetp. | |
| Additional | Requirement to give | Possible requirement |
| intervention. | subject a substance to | to immune suppress |
| | induce provirus release | subject to prevent |
| | and demise of intra- | immune system inter- |
| | cellular parasite | ference with thera- |
| | gene delivery vector. | peutic or graft |
| | | verses host disease. |

In its simplest form a sacromastigophoric organism contains a recombinant lytic factor gene under the control of an exogenous species promoter. In the exemplary case of a trypanosome, the lytic factor is preferably Hpr and the exogenous species promoter is an antibiotic inducible promoter such as tetracycline inducible promoter. Alternative lytic factors encode trialysin, a pore forming protein from Triatoma infestans (Amino et al., 2002), or the catalytic domain of diphtheria or pseudomonas exotoxin A (vanderSpek et al., 1994; Debinski et al., 1995) or apoptotic proteins Bad or Bax. With the induction of the promoter, organism lysis occurs and results in the host being exposed to the organism components thereby triggering an immune response to an otherwise undetected organism. As the genome and proteins of the pathogenic sacromastigophoric organisms is known (Porcel et al., 2000), this represents an operative mode of vaccination against a variety of protozoa diseases for which no vaccine currently exists.

One utility of the invention is the delivery of a protein. The inventive cell-based delivery system can produce the protein, fold it correctly and modify it appropriately, prior to release from the cell. Provided that the immune system is evaded, this type of drug delivery offers greater longevity and the ability to continually deliver material such that a persistent level of therapeutic agent is produced.

The present invention also includes the design of an inducible multiple gene expression vector that expresses the genes required for targeted homologous recombinant event and demise of the delivery sacromastigophoric organism. In the prototype T. brucei, a multiple gene expression vector is designed with trypanosome inducible promoters (T7p). The expression cassette contains a promoter; ribosome binding site sequence for instances where polypeptide expression is desired; restriction sites for cloning therein genes of interest, and a T7 termination signal. The T7 RNA polymerase promoter is known in the art to work in the trypanosome and is therefore used to drive gene expression. The plasmid is designed for cassette gene expression. The cassette design facilitates insertion of various transposable elements. The two exemplary genetic elements are a modified retroviral vector and haptoglobin related protein.

In an exemplary embodiment of an expression cassette, a construction of the inducible vector promotes the expression of a transposon (provirus) for host genome integration and demise of the sacromastigophoric organism. The inducible genetic elements that drive host gene integration and trypanosome lysis are a modified retrovirus and haptoglobin related protein. The modified retrovirus is a replication incompetent RNA virus genome. This element contains the LTRs and enzymes reverse transcriptase and integrase. These elements facilitate gene integration of a low-toxicity green fluorescent protein (hrGFP, Stratagene). GFP gene expression will be under control of a promoter involved in angiogenesis. This promoter is activated during angiogenesis. The integration of this construct results in a cell line that has an angiogenesis inducible reporter has utility as a model for angiogenesis. This model is useful for high throughput screening for compounds with agonistic and antagonistic angiogenic properties.

The haptoglobin related protein has been reported to induce apoptosis in Trypanosoma brucei brucei. The induction of apoptosis-like phenomena in the trypanosome after cell invasion results in eradication of the organism from the host and release of the expressed construct DNAs or non-nucleic acid therapeutic agents. Other suitable apoptotic proteins illustratively include trialysin, Bad and Bax.

The cell biology of trypanosomes provides two mechanisms that are utilized to engineer tetracycline sensitive Hpr lysis. Trypanosomes are polarized elongated cells with a single flagellum that emerges from a posterior pocket. Because of the packing of subpellicular microtubules beneath the plasma membrane the flagellar pocket is the only external membrane region that is available for vesicular transport. The flagellar pocket is essential for the bloodstream parasite to export major coat protein and import host-derived macromolecules. The flagellar pocket is used as an entry site for proteins required for normal trypanosome biology. For example p67, a lysosomal membrane glycoprotein, is synthesized and exported to the flagellar pocket whereby it is subsequently endocytosed. Following endocytosis p67 is targeted to the lysosome (Alexander et al., 2002). In the non-bloodstream procyclic form of trypanosome p67 is directly transported from the Golgi to the lysosome (Kelley et al., 1995). p67 is directed to the lysosome via two pathways depending on the developmental stage of the trypanosome making it is possible to use the signaling sequences that target p67 to control the trafficking of Hpr.

Experimental determination of the efficiency of gene transfer between an inventive organism and a eukaryotic host is provided using in vitro tissue culture models. The modified retroviral vector or transposon is integrated into the eukaryotic cell host genome and detectable amounts of the organisms are eliminated in in vitro cultures. Genomic integration of engineered transposon results in the expression of GFP after induction with angiogeneic compounds. GFP expression is monitored spectrophoretically. The stable transfected cell line acts as a model for angiogenesis with a fluorescent reporter. This cell line is an operative model for screening compounds with anti-angiogeneic and hence anti-tumor activities. The trypanosome vector is readily modified to deliver up to three proviral constructs, each of which may contain two genes. Further engineering of the trypanosome gene delivery system for specific receptor targeting is also appreciated to be an aspect of the present invention for modifying the specificity of an inventive organism.

Non-nucleic acid therapeutic agents are packaged in a sacromastigophoric organism through electroporation or phagocytosis of liposomally packaged therapeutic agents. Representative liposomal packaging processes operative in concert with the present invention are detailed in U.S. Pat. Nos. 4,356,167; 4,873,088; and 5,843,475. Unlike nucleic acid therapeutic agents which replicate in concert with organism reproduction, it is appreciated that non-nucleic acid therapeutic agents must be integrated into an inventive organism and the organism administered in a therapeutic amount.

In a preferred embodiment, the expression cassette also incorporates proximal to the lytic factor gene, a gene that confers antibiotic resistance to an otherwise effective antibiotic against the native sacromastigophora protozoa. More preferably, the antibiotic resistance conferring gene is under regulatory control of the same exogenous RNA polymerase used to repress the lytic factor in the absence of the exogenous species that induces the lytic factor. Bacterial T7 RNA polymerase (T7p) is a representative control element. The inclusion of an antibiotic conferring gene serves to screen for those individual organisms having a functional recombinant lytic factor.

Molecular biological tools and techniques have been developed that can be used to genetically engineer T. bruei (Wirtz et al., 1999; Epicentre, Inc., Madison, Wis.). An inducible system has been developed for controlling target gene expression in T. brucei brucei. The system uses bacterial T7 RNA polymerase (T7p), the tetracycline repressor (TetR) and a trypanosome promoter that has been modified to contain the tetracycline operator, making target gene expression responsive to tetracycline (Morris et al., 2001; Biebinger et al., 1997, Wirtz et al., 1999). Subsequently, transposon mutagenesis techniques have been developed and commercialized (Epicentre, Inc.) for T. brucei brucei. These techniques allow for easy genetic manipulation and engineering of the organism (Goryshin et al., 2000; Shi et al., 2002).

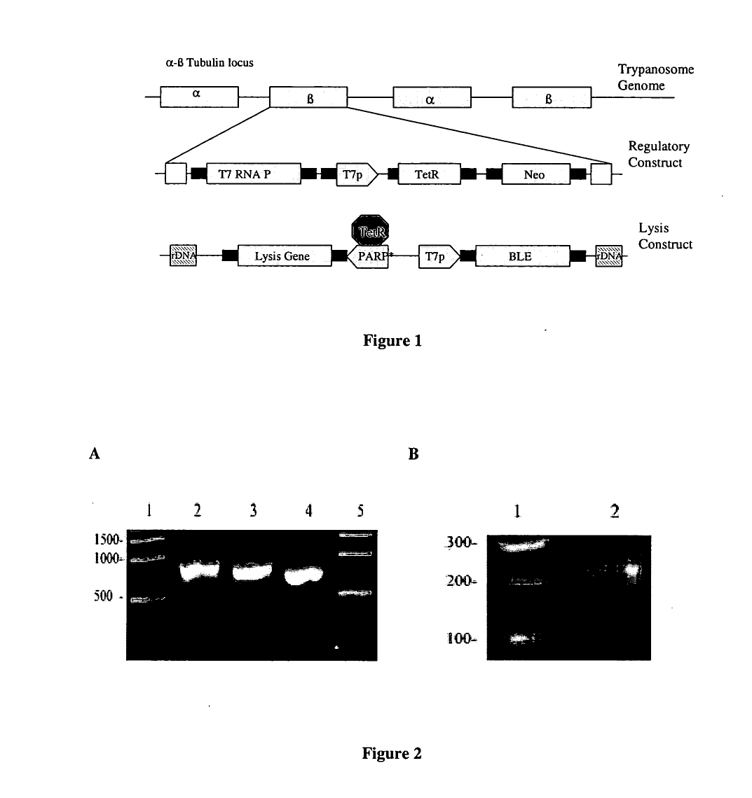

By way of example, a single-marker Trypanosoma brucei cell line has the genes for T7 RNA polymerase, tetracycline repressor, and neomycin phosphotransferase integrated into its genome as shown in FIG. 1. The construct was integrated into the β tubulin locus by homologous recombination. The tubulin promoter drives constitutive expression of T7 RNA polymerase. A modified T7 promoter drives Tet repressor expression. The construct confers neomycin resistance. Performing a second homologous recombination event generates tightly regulated inducible target gene expression. The “single-marker” line contains the regulatory construct (middle) containing T7 RNA polymerase (T7 RNA P), Tet repressor (TetR) and neomycin phosphotransferase (Neo) genes integrated into the tubulin locus at the top of FIG. 1 by homologous recombination. Also depicted in FIG. 1 is the lysis construct containing the target gene(s) under control of Tet inducible promoter (PARP*) and a gene conferring resistance to phleomycin (BLE).

In the absence of tetracycline the TetR binds to the modified PARP* promoter blocking expression of the lysis gene. Upon introduction of tetracycline the TetR is removed from its binding site on the PARP* promoter allowing expression of the lysis gene. Previous work has shown that this inducible system responds to the level of tetracycline resulting in expression that varies over a 10³-10⁴ -fold range (Wirtz et al., 1999).

The present invention is further illustrated through the following non-limiting examples.

### EXAMPLE 1

Multi-gene expression vectors are initially designed for expressing viral capsid proteins. An expression vector with four echovirus-1 (EV1) capsid genes was constructed with each under control of an individual T7p/lac/p10 promoter cassette. Four genes were extracted and used to construct a phage. This required the genetic engineering of a unique expression system capable of independently expressing four gene targets. This expression system was derived from a commercial vector that is operative in both eukaryotic and prokaryotic systems. Subsequent to the completed construction of the expression vector, the EV1 capsid genes were subcloned. A unique expression system was partially constructed. This expression system contains three of the four target (viral) genes each individually under the control of a T7p/lac/p10 cassette promoter. The construct contains a unique signature security sequence.

Production of the Trojan Phage involved design of first generation viral prototype; synthesis of required nucleic acids with the synthesis and cloning of pieces of nucleic acid; construction and sequencing of initial viral prototypes, where enzymatic manipulations of nucleic acids were used to piece the phage system together in a modified expression vector; and demonstration and validation of correct viral capsid protein (VP) expression.

Between the EV1 two coat proteins VP1 and VP4, there appears to be three regions that could accommodate the insertion of a foreign protein. These regions form the basis for the construction of three inventive crystallization systems. The VP1 and VP4 subunits were selected because they have amino or carboxyl termini that are presented on the interior surface of the viral capsid and are located near the center of the protomer complex. The protomer complex is a triangular-shaped assembly composed of the four viral coat proteins VP1, VP2, VP3 and VP4. Twenty protomer complexes self assemble to construct the viral capsid or protein shell. It is appreciated that selecting termini that are located in the central portion of the protomer unit will have the least impact on protomer and capsid assembly.

A commercially available dual system expression vector, pTriEx-1.1 (Novagen, Inc.), was selected as the parental plasmid for the construction of a multi-gene expression plasmid that drives the phage. The life cycle of EV1 occurs in eukaryotic cells. A characteristic of the picomavirus is that the single strand RNA genome is translated into a single polyprotein. This polyprotein is then enzymatically cleaved into functional units. Therefore, to construct a recombinant phage expression system it was decided that a modular polycistronic-like expression system was required. A similar expression system was constructed, by Dr. Song Tan, and demonstrated to function in prokaryotic systems (Tan, Song 2001). Dr. Tan's system used a single promoter (T7p) to drive the transcription of a single mRNA with multiple ribosomal binding sites that drove multiple gene expression. The expression system used herein has four independent promoters that produce four mRNA species that each encode for the functional viral capsid molecules and using four independent promoters generates basically equivalent amounts of message for each gene. In contrast, the use of a polycistronic system cannot guarantee equal expression of multiple genes. The vector, pTriEx-1.1, was selected for genetic modification to express four genes because it has the control elements for protein expression in both prokaryotic and eukaryotic systems. A modular system is a prerequisite because expression of poly-proteins in E. coli results in the formation of inclusion bodies, whereas cleavage into function units resulted in recapitulation of viral capsids (Lewis et al. 1991). The ability to move the whole pTriEx-1.1 derived system into a eukaryotic host was attractive in the event that the bacterial system failed to produce assembled capsids due to improper protein folding. The system developed in this work is different than the one developed by Dr. Tan in that prokaryotic (T7p/lac) and eukaryotic insect cell (p10) promoters are used to drive the expression of each individual gene. These promoters also include a ribosome-binding site (RBS) for bacterial expression (Tan, S, 2001).

The genes that encode for echovirus 1 proteins VP1, VP2, VP3 and VP4 were extracted by RT-PCR. Echovirus 1 was propagated and partially purified as described (Filman et al. 1998). The RNA genome was purified from the viral preparation using TRIzol LS (Life Technologies). The purified viral RNA was subjected to RT-PCR using oligonucleotides TP7-14 as shown in Table 2.

TABLE 2

Oligonucleotides used for sequencing and subcloning

| | | |
|---|---|---|
| Name | Sequence* | Purpose |

| | | |
|---|---|---|
| TP1 | 5′AATTATTCGAATTAGCAAGAAGATATTGTACCGAAATTAATACGACTCACTATAGGGG′3 (SEQ ID NO. 1) | VP1p |
| TP2 | 5′TAAATAAAGCGGCC/GCTTATTACTAG/TCTTTATCTCCTTTGATTGTAAATAAAATGTAATTTACAGTAT′3 | VP1p |
| | (SEQ ID NO. 2) | |
| TP3 | 5′ATAGCATGGTAC/CACCGAAATTAATACGACTCACTATAGGGG3′ (SEQ ID NO. 3) | VP3p |
| TP4 | 5′AATACTAGTT/CGAAGGTAGGTAGCTAGCGTATATCTCCTTTGATTGTAAATAAAATGTAATTTACAGTAT′3 | VP3p |
| | (SEQ ID NO. 4) | |
| TP5 | 5′ATATTAGG/CGCGCCACCGAAATTAATACGACTCACTATAGGGG3′ (SEQ ID NO. 5) | VP2p |
| TP6 | 5′ATTAATCTGCAGATTTATAGGCGC/CGTATATCTCCTTTGATTGTAAATAAAATGTAATTTACAGTAT′3 | VP2p |
| | (SEQ ID NO. 6) | |
| TP7 | 5′GCTTCA/CTAGTTCTGACTGC/TAAGCATGGGTGATGTGCAGAATGCTGTCG3′ (SEQ ID NO. 7) | VP1s |
| TP8 | 5′CAAGGTT GCGGCC/GCGATGATCGTTGTTATTATGTTGG3′ (SEQ ID NO. 8) | VP1s |
| TP9 | 5′GCTTCG/CTAGCATGGGACTACCGACCATGAACACCCCTGGC3′ (SEQ ID NO. 9) | VP3s |
| TP10 | 5′CGGCCATT/CGAACTACTATTGGTAAAAAGATGTTTGCTC3′ (SEQ ID NO. 10) | VP3s |
| TP11 | 5′GCTTCGG/CGCCATGTCTCCAACGGTTGAAGAGTGC3′ (SEQ ID NO. 11) | VP2s |
| TP12 | 5′GCCGGAACTGCA/GCTACTATTGGTGTCCAGCTAGTCG3′ (SEQ ID NO. 12) | VP2s |
| TP13 | 5′GCTTCAGAT/ATCGGTATCAACACAGAAGACCGGGGCGCAC3′ (SEQ ID NO. 13) | VP4s |
| TP14 | 5′GCCATCCGGAGATC/TCTACTACG/AATTCAGCGCTGGCAGGGTTTTTATCATGACATCTTTCATTGGTTCAGT | VP4s |
| | AAACTTCCC/GGG GTCTTGGGTGAA3′ (SEQ ID NO. 14) | |
| TPIL7spe1 | 5′GCGCGA/CTAGTCTGATGGACTGCGACATCG3′ (SEQ ID NO. 15) | TGs1 |
| TPIL7bpu1 | 5′CACCCATGCTTAGCGCGTGTTCTTTAGTGCCCATC3′ (SEQ ID NO. 16) | TGs1 |
| 102I | | |
| TPIL7avri | 5′GCTCGCCC/GGGATGGACTGCGACATCG3′ (SEQ ID NO. 17) | TGs2 |
| TPIL7bglii | 5′GCCATCCGGA/GATCTCTAGTGTTCTTTAGTGCCC3′ (SEQ ID NO. 18) | TGs2 |
| TPIL7ecori | 5′GCGCTG/AATTCGATGGACTGCGACATCG3′ (SEQ ID NO. 19) | TGs3 |

*The T7/lac control element is underlined. Security signature sequence in TP1 is displayed in italics.

An improved Superscript one-step RT-PCR system (Invitrogen) with oligonucleotide pairs synthesized to flank viral structural genes was used to extract the VP1, VP2, VP3, and VP4 sequences. Oligonucleotide pair TP7 (SEQ ID NO. 7)/TP8 (SEQ ID NO. 8) were used to generate the gene VP1 with unique restriction sites SpeI, Bpu 1102 I, and Not I, for vectorial cloning. Similarly oligo pairs TP9 (SEQ ID NO. 9)/TP10 (SEQ ID NO. 10), TP11 (SEQ ID NO. 11)/TP12 (SEQ ID NO. 12), and TP13 (SEQ ID NO. 13)/TP14 (SEQ ID NO. 14) were used to generate VP3, VP2, and VP4 with unique restriction sites NheI/NspV, NarI/Pstl, and EcoRV/BglII, respectively. The RT-PCR reaction generated products with electrophoretic mobility comparable to the predicted product sizes as shown in FIGS. 1A and 1B. In FIGS. 1A and 1B, all RT-PCR reactions were performed using the purified native EV 1 genome as the template. PCR products were visualized in an agarose gel stained with ethidium bromide under UV light. Panel A: Lanes 1 and 5: Molecular weight markers (bp). Lane 2: The VP1 product (891 bp) generated using oligonucleotides TP7 and TP8. Lane 3: The VP2 product (815 bp) generated using oligonucleotides TP11 and TP12. Lane 4: The VP3 product (748 bp) generated using oligonucleotides TP9 and TP10. Panel B: Lane 1: Molecular weight markers (bp). Lane 2: The VP4 product (239 bp) generated using oligonucleotides TP13 and TP14. The RT-PCR amplification of EV1 viral protein structural genes VP1s, VP2s, and VP3s were performed in a similar manner as VP4s with one exception: capsid structural genes VP1, VP2 and VP3 were amplified with primers that introduced an initial ATG translation start codon. The RT-PCR primers have unique restriction sites that facilitate vectorial cloning.

Three T7/lac, p10 and RBS transcription/translation cassettes from pTreEx-1.1 were amplified using primer pairs TP1 (SEQ ID NO. 1)/TP2 (SEQ ID NO. 2), TP3 (SEQ ID NO. 3)/TP4 (SEQ ID NO. 4), and TP5 (SEQ ID NO. 5)/TP6 (SEQ ID NO. 6). Flanking sequences of the primers contained unique restriction sites that facilitated vectorial cloning back into pTriEx-1.1. PCR performed on pTriEx-1.1 with primer pair TP1 (SEQ ID NO. 1)/TP2 (SEQ ID NO. 2) generated a genetic control element with NspV and Not I restriction sites for driving VP-1 gene expression. Similarly PCR procedure performed with primer pairs TP3 (SEQ ID NO. 3)/TP4 (SEQ ID NO. 4) and TP5 (SEQ ID NO. 5)/TP6 (SEQ ID NO. 6) produced control elements with unique Kpn I/Nsp V and Asc I/Pst I restriction sites for vectorial cloning back into pTriEx-1.1 for driving gene expression of VP-3 and VP-2, respectively, as shown in FIG. 2. In FIG. 2, all reactions used pTriEx-1.1 as the template. Lane 1: molecular weight markers (bp). PCR products were visualized in a 4% agarose gel stained with ethidium bromide under UV light. Lane 2: T7lac and p10 promoter operator (245 bp) for VP1 generated using oligonucleotides TP1 and TP2 (PCR1). Lane 3: T7lac and p10 promoter operator (230 bp) for VP3 generated using oligonucleotides TP3 and TP4 (PCR2). Lane 4: T7lac and p10 promoter operator (228 bp) for VP2 generated using oligonucleotides TP5 and TP6 (PCR3). The PCR product from primer pair TP1 (SEQ ID NO. 1)/TP2 (SEQ ID NO. 2) also contains a unique signature security sequence, when translated into the one letter amino acid residue code, spells LARRYC. VP-4 gene expression is driven by the original control elements in pTriEx- 1.1.

The sequence of DNA primers used in this work was based on sequence information provided by Genebank and Novagen, Inc. The design of DNA primers for RT-PCR amplification of echovirus 1 capsid genes was based upon the published sequence deposited in Genebank (AF029859). PCR primers designed for duplicating the tri promoter cassette in pTriEx-1.1 were based on the sequence from Novagen, Inc. accessible through the Internet (http://www.novagen.com/). All DNA primers were ordered from the Oligonucleotide Synthesis Core Facility at the University of Alabama at Birmingham, Comprehensive Cancer Center.

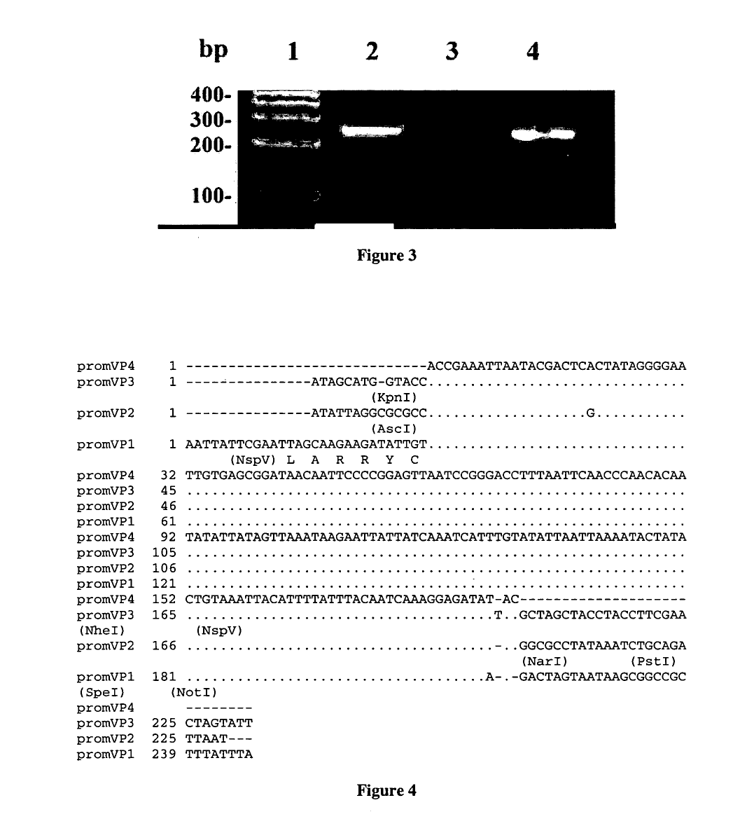

Four echovirus 1 capsid genes were cloned under control of T7p/lac/p10 promoter cassette. PCR products containing genetic control elements for driving gene expression were digested with the appropriate endonucleases as described by the manufacturers. Visual inspection of digested and non-digested PCR fragments was performed to insure that the reactions worked. Digested fragments were then gel purified and used for ligation reactions. The fragments of DNA encoding for T7/Lac/p10 cassette promoters that were amplified as shown in FIG. 3, cloned and sequences are compared to the original promoter region in pTriEx-1.1, sequence identification shown on the left. Differences between the template and duplicated DNA fragments are displayed. Engineered restriction sites are indicated in parenthesis. A unique security sequence and its one letter amino acid sequence for the VP1 promoter is also displayed.

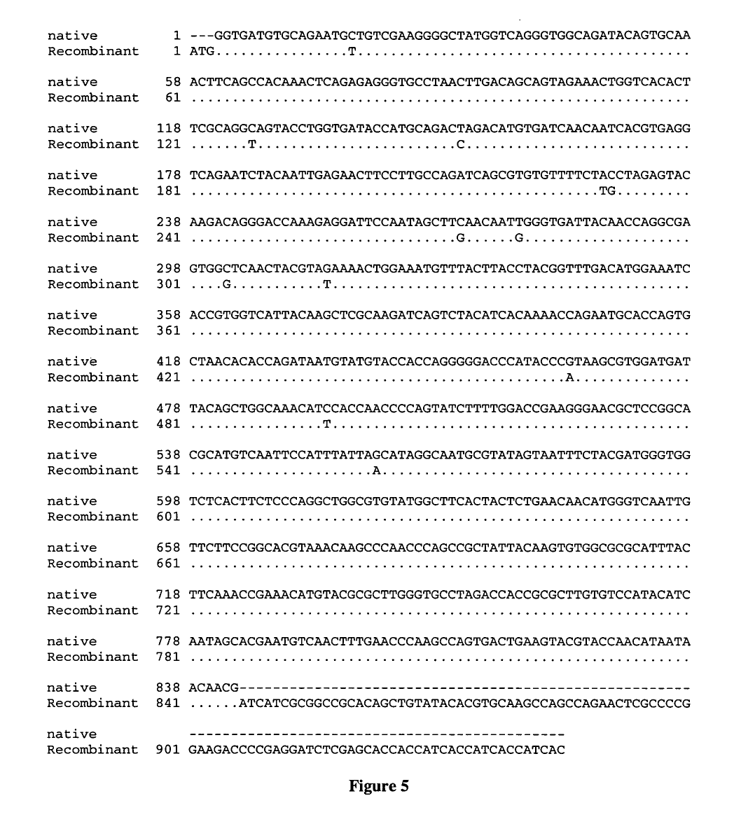

FIG. 5 shows the recombinant DNA fragment that encodes for the echovirus 1 VP1 shell protein (SEQ ID NO. 20) extracted by RT-PCR as shown in FIG. 2 following cloning and sequencing. The native gene (SEQ ID NO. 21) is displayed for comparison.

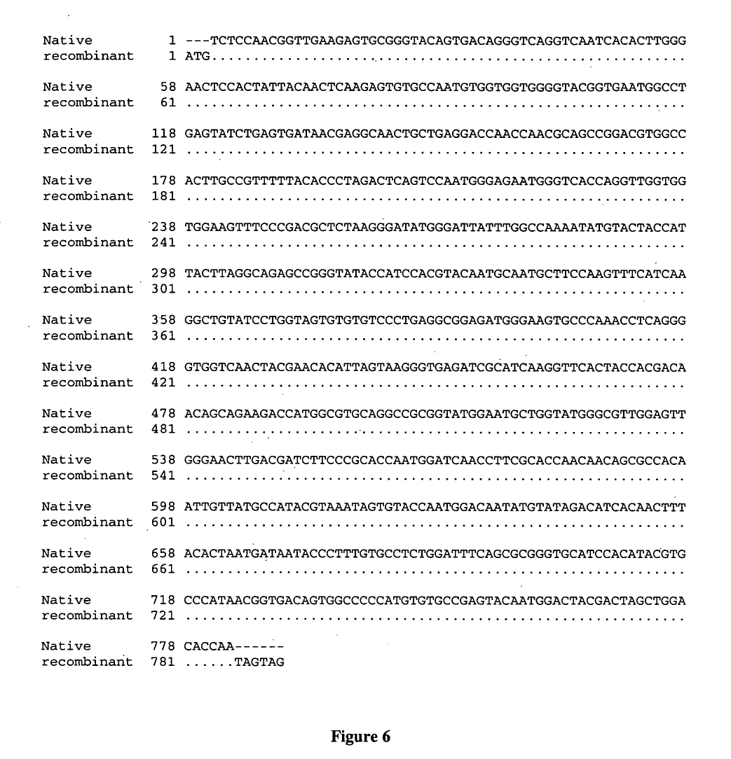

FIG. 6 shows the recombinant DNA fragment that encodes for the echovirus 1 VP2 shell protein (SEQ ID NO. 22) extracted by RT-PCR as shown in FIG. 2 following cloning and sequencing. The native gene (SEQ ID NO. 23) is displayed for comparison.

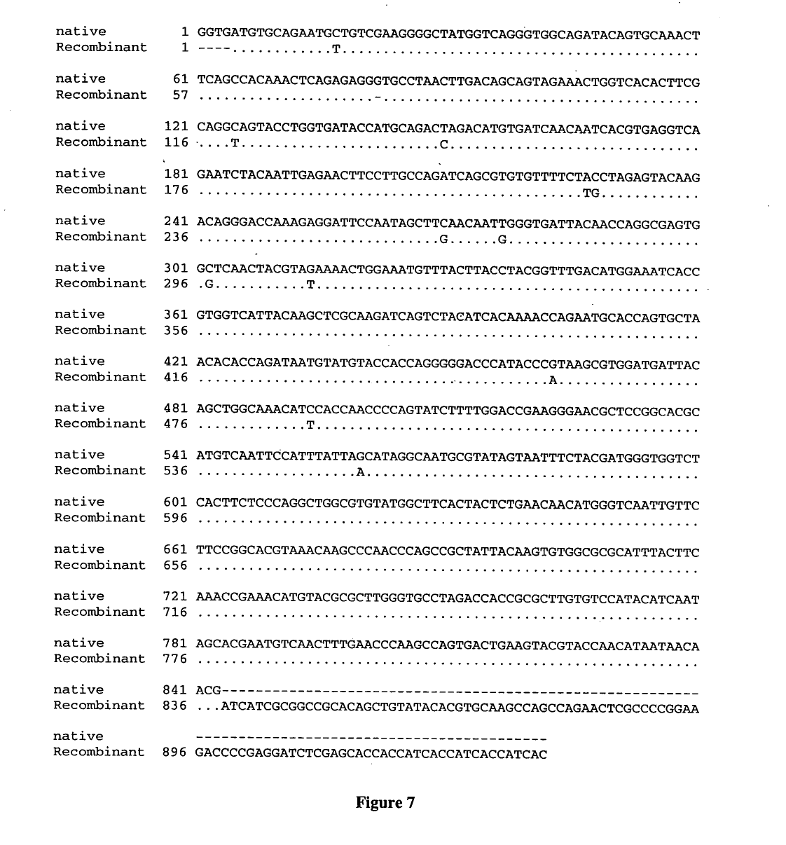

FIG. 7 shows the recombinant DNA fragment that encodes for the echovirus 1 VP3 shell protein (SEQ ID NO. 24) extracted by RT-PCR as shown in FIG. 2 following cloning and sequencing. The native gene (SEQ ID NO. 25) is displayed for comparison.

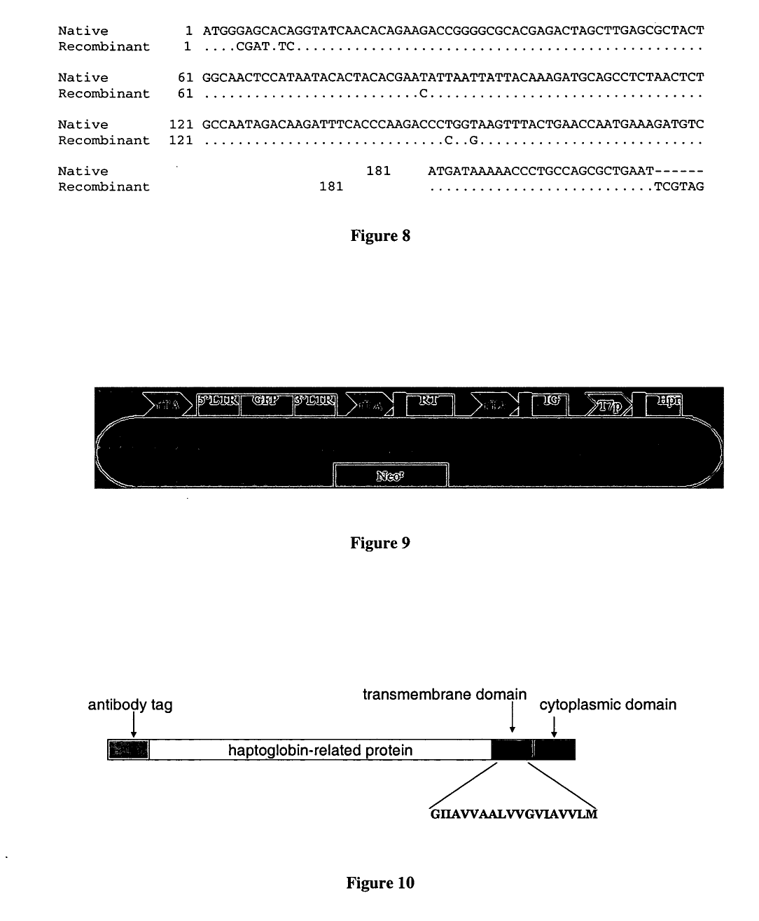

FIG. 8 shows the recombinant DNA fragment that encodes for the echovirus 1 VP4 shell protein (SEQ ID NO. 26) extracted by RT-PCR as shown in FIG. 2 following cloning and sequencing. The native gene (SEQ ID NO. 27) is displayed for comparison.

The smallest capsid gene VP4 was directly cloned into pTriEx-1.1 under control of the T7p/lac/p10 promoter to form pLCVP4. The three capsid genes, VP1-3, and their individual promoters were constructed in parallel and then sequentially cloned into pLCVP4. As each capsid gene was cloned into the expression vector it was sequenced to insure proper nucleotide incorporation.

The smallest capsid gene, VP4, was cloned first using EcoRV and BglII to produce pLCVP4. The VP4 structural (VP4s) gene was cloned using RT-PCR. TP13 (SEQ ID NO. 13) and TP14 (SEQ ID NO. 14) are the primers and the EV1 RNA genome is the template. The VP4s contains a unique Eco RI restriction site that was maintained. By keeping the native Eco RI restriction site a serine residue is added onto the carboxyl terminal of the VP4 protein. The original T7/lac and p10 operators present in the pTriEx-1.1 vector drive VP4 gene expression. VP4 is a unique opportunity because of the possibility of forming chimeric molecules with the target protein in the carboxyl terminal half of VP4. This helps in subcloning because it eliminates the necessary removal of target gene stop codons. and thus creates an opportunity for the insertion of multiple target genes simultaneously. A unique Avr I restriction site was introduced into the VP4s to allow for creating chimeric molecules with a linker region that starts at VP4₅₁. The Avr I restriction site modifies the native VP4s sequence by introducing silent mutations. The silent mutations alter the native nucleic acid sequence (CCT GGT) to (CCC GGG) but will not change the protein composition (Pro Gly). The recombinant VP4s was cloned into the pTriEx-1.1 vector using the Eco RV and Bgl II unique restriction sites. By cloning into the Eco RV restriction site we pick up the initiator methionine (M), alanine (A) and serine (S). This will mutate the native VP4 amino terminal sequence MGAQ to MAIS. The VP4s cloning task was completed by DNA sequencing to insure proper nucleotide incorporation and that the gene was cloned in frame with the initial ATG translation start sequence as shown in FIG. 8. The VP4s encodes for a protein with a predicted molecular weight of 6800 Da.

The three promoter sequences synthesized by PCR were used to construct three plasmids that would accept capsid genes VP1, VP2 and VP3. These promoter sequences synthesized were individually cloned in parallel into pTriEx-1.1. This strategy produced three expression vectors ready to accept the viral capsid genes VP1, VP2, and VP3. PCR1 was vectorially cloned into pTriEx-1.1 using NspV and NotI. Genetic control elements PCR2 and PCR3 were likewise cloned into pTriEx-1.1 vectors using restriction sites (AscI, PstI) and (KpnI, NspV), respectively. The resultant dual promoter cloning products were sequenced to insure that proper DNA sequence was maintained. The capsid genes VP1-3 were digested with the appropriate enzymes and then cloned in parallel into the new expression vectors under control of the synthesized promoters to produce plasmids pLCVP1, pLCVP2, and pLCVP3. The capsid genes were also sequenced to insure proper nucleotide incorporation was maintained during the RT-PCR. During the RT-PCR there were numerous nucleotide mis-incorporations as shown in FIGS. 5-7. Many of these mistakes altered the DNA sequence of the gene but did not change the amino acid sequence. Nucleotide mis-incorporations that resulted in point mutations were observed in capsid genes VP1 and VP3. DNA sequencing of the VP1 gene demonstrated six mutations. Two point mutations were observed in the VP3 gene that altered the predicted amino acid sequence. These two point mutations encoded for the substitution of tyrosine 70 with histidine and serine 113 with leucine. Conventional laboratory techniques are employed to mutate these sequences to encode for the native amino acid residues.

An inventive multiple gene expression vector was partially constructed by the sequential cloning of individual viral capsid genes along with their promoters into pLCVP4. The promoter region and VP3 structural cassette was the first to be cloned into pLCVP4 using restriction sites AscI and PstI to produce pLCVP4-VP3. The VP1 cassette was vectorially cloned into pLCVP4-VP3 next using NspV and NotI. The sequential insertion of each VP expression cassette was verified by restriction digestion and agarose gel analysis. The genes for VP3 and VP1 encode for the production of protein with molecular weight of 26000 Da and 31000 Da, respectively.

### EXAMPLE 2

An inducible multiple gene expression vector is designed to express the genes required for targeted homologous recombinant event and lysis of the delivery organism. A multiple gene expression vector is designed with trypanosome inducible promoters (T7p). A dual promoter plasmid construct is designed and constructed. The expression cassette contains a promoter, ribosome binding site sequence, restriction sites for cloning genes of interest, and T7 termination signal. The T7 RNA polymerase promoter has been demonstrated to work in the trypanosome and drives gene expression. The plasmid is designed for cassette gene expression. The cassette design facilitates various transposable element exchanges. The two genetic elements detailed herein are a modified retroviral vector and haptoglobin related protein (Hpr).

Design of tLCRC:

The tLCRC controls the expression of four target DNAs: integration component, homologous recombinant provirus, reverse transcriptase, and releasing factor. The design of tLCRC begins with a plasmid expression vector, pMaGEX, has been constructed to express multiple gene cassettes. The expression vector pMaGEX contains the genetic control elements for inducible expression of four target genes. The elements that drive gene expression are composite promoters containing the DNA sequence recognized by T7 RNA polymerase, lac operon proteins and p10 transcription machinery. An integration component contains the gene(s) required for a homologous recombinant event. This element produces mRNA with ribosome binding site (RBS) and result in the protein production of molecules responsible for target gene integration into a host genome. The provirus is a genetic element that contains flanking LTR sequences for host site-specific integration, a reporter gene driven by an inducible promoter, and a selection marker. The reporter gene encodes the green fluorescent protein and will be designed for cassette replacement. Cassette replacement criterion facilitates the exchange of reported DNA for gene(s) of therapeutic value. Reverse transcriptase is used to act on the engineered provirus to result in the synthesis of multiple copies of linear DNA for host genome integration. It is anticipated that multiple copies of the engineered provirus increase the likelihood of host genome integration. The releasing factor is a gene under control of a tetracycline sensitive promoter that encodes for a haptoglobulin-related protein, Hpr, reported to be involved in the apoptosis of Trypanosoma brucei. It is anticipated that initiation of apoptosis of the Trypanosoma after host cell infection will destroy the parasite and release the expression vector without harming the host cell. A diagram of the vector is shown in FIG. 9.

It is anticipated that chemical induction of the trypanosome lytic factor, haptoglobin-related protein, will release the plasmid from the intracellular parasite into the host cell whereby homologous recombination can be activated by the addition of tetracycline.

### EXAMPLE 3

Construction of the inducible vector promotes the expression of a transposon (provirus) for host genome integration and demise of the trypanosoma organism. The inducible genetic elements that drive host gene integration and trypanosome apoptosis are a modified retrovirus and haptoglobin related protein. The modified retrovirus is a replication incompetent RNA virus genome. This element contains the LTRs and enzymes reverse transcriptase and integrase. These elements facilitate gene integration of a low-toxicity green fluorescent protein (hrGFP, Stratagene). GFP gene expression is under control of the Tiel promoter. This promoter is activated during angiogenesis. This construct yields a cell line that has an angiogenesis inducible reporter that could be used as a model for anti-angiogenesis compounds.

Since the site of action for Hpr is the lysosome of the trypanosome, the Hpr gene used herein contains a lysosomal targeting sequence. Alexander et al. have shown that a 43 amino acid lysosomal-targeting sequence was sufficient to cause a heterologous protein, green fluorescent protein, to be localized to the lysosome of procyclic trypanosomes. (Alexander et al., 2002). In order to evaluate the subcellular localization of Hpr a short antibody tag is also incorporated to allow immune fluorescent microscopy of cells expressing Hpr as shown in FIG. 10. Trafficking of Hpr is monitored by immune histochemistry techniques. FIG. 10 shows the coding sequence for haptoglobin-related protein is indicated as are the N-terminal antibody tag and a bipartite C-terminal sequence for targeting to the lysosome.

### EXAMPLE 4

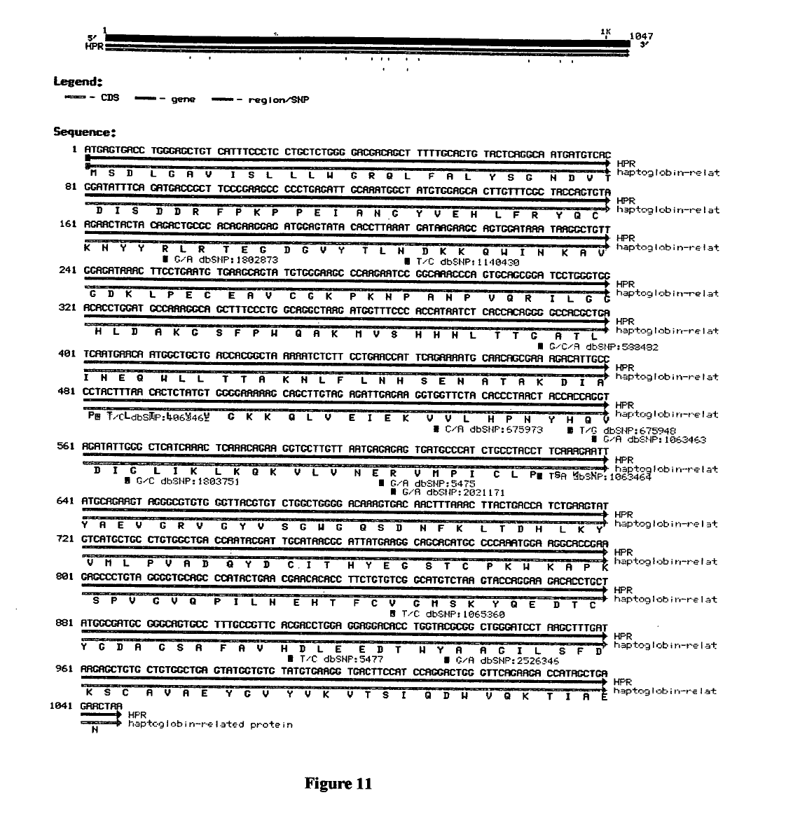

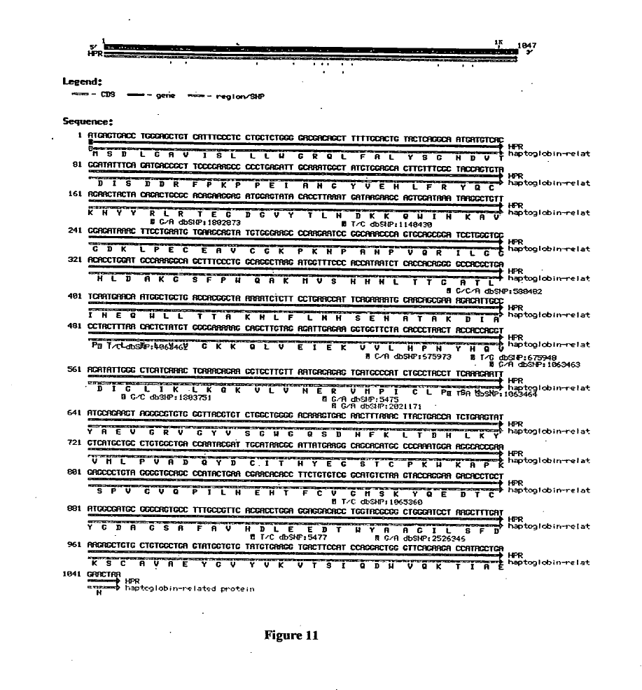

Haptoglobin-related protein (Hpr) is the toxin to induce lysis of Trypanosoma brucei. A clone containing the known Hpr gene coding sequence (SEQ ID NO. 28) has been generated as shown in FIG. 11. FIG. 11 also includes the amino acid sequence (SEQ ID NO. 29) encoded by the gene, as well as the codon and amino acid polymorphisms known to exist therein. A set of primers to preferentially recognize the Hpr sequence was designed and tested. The primers were shown to preferentially amplify Hpr DNA as confirmed by restriction enzyme analysis. The DNA probe generated using these Hpr primers, was used to screen an expression library from a human hepatic carcinoma cell line (HepG2). A number of positive clones were identified and sequenced. This work revealed that approximately one third of the clones represented Hpr. Both the Hpr and Hp coding sequences were subcloned into a plasmid vector for ease of manipulation. Plasmid containing Hpr is used to generate Hpr with a lysosomal targeting sequence and antibody tag to track and confirm localization when expressed in trypanosomes.

### EXAMPLE 5

Experimental determination of gene transfer between an inventive organism and a eukaryotic host is performed in vitro. Trypanosome infected human myocardial cells in culture display inducible fluorescence. The modified retroviral vector or transposon integrates into the eukaryotic cell host genome and all trypanosome organisms eliminated through administration of a 10 mg/kg/day solution of tetracycline to the culture. Genomic integration of engineered transposon results in the expression of GFP after induction. GFP expression is monitored spectrophoretically by known techniques. The stable transfected cell line acts as a model that fluorescently reports back. This cell line is a model with applications for screening compounds for anti tumor activities. The trypanosome gene delivery vector will be modified to deliver up to three transposon constructs, each of which may contain two genes. Further engineering of the trypanosome gene delivery system for specific receptor targeting is also proposed.

### EXAMPLE 6

To test the in vivo efficacy of the present invention, a group of healthy Sprague-Dawley mice are parenterally injected with an engineered Trypanosoma brucei brucei organism containing a tetracycline responsive lytic factor gene as detailed above. Seventy-two hours post infection, the mice are given daily doses of tetracycline over a period of 72 hours. Blood titers are performed for the delivery organism at 24-hour intervals beginning just prior to tetracycline administration. Similar experiments using recombinant Trypanosoma cruzi that is capable of entering host cells allow for the spectroscopic observation of GFP expression subsequent to organism lysis through tetracycline exposure.

### LITERATURE CITED

Alexander, D. L., Schwartz, K. J., Balber, A. E. and Bangs, J. D. (2002) Developmentally regulated trafficking of the lysosomal membrane protein p67 in Trypanosoma brucei. J. Cell Science 115(16):3253-3263.
Amino R, Martins R M, Procopio J, Hirata I Y, Juliano M A, Schenkman S. (2002) Trialysin, a novel pore-forming protein from saliva of hematophagous insects activated by limited proteolysis. J Biol Chem. 277(8):6207-13. Epub 2001 December.
Biebinger S, Wirtz L E, Lorenz P, Clayton C. (1997) Vectors for inducible expression of toxic gene products in bloodstream and procyclic Trypanosoma brucei. Mol Biochem Parasitol. 85(1):99-112.
Bishop, J. R., Shimamura, M., and Hajduk, S. L. (2001). “Insight into the mechanism of trypanosome lytic factor-1 killing of Trypanosoma brucei brucei.” Molecular & Biochemical Parasitology, 118, 33-40.
Damian, R. T. (1997). “Parasite immune evasion and exploitation: Reflections and projections.” Parasitology, 115 (Suppl.), S169-S175.
Debinski, W., Obiri, N. T., Pastan, I. and Puri, R. K. (1995) A novel chimeric protein composed of interleukin 13 and Pseudomonas exotoxin is highly cytotoxic to human carcinoma cells expressing receptors for interleukin 13 and interleukin 4. J. Biol. Chem. 270(28):16775-16780.
Filman, D. J., Wien, M. W., Cunningham, J. A., Bergelson, J. M. And Hogle, J. M. Structure determination of echovirus 1. Acta. Cryst. 1998, D54, 1261-1272
Furger, A., Jungi, T. W., Salomone, J. -Y., Weynants, V., and Roditi, I. (2001). “Stable expression of biologically active recombinant bovine interleukin-4 in Trypanosoma brucei.” FEBS Letters, 508, 90-94.
Goryshin I Y, Jendrisak J, Hoffman L M, Meis R, Reznikoff W S. (2000) Insertional transposon mutagenesis by electroporation of released Tn5 transposition complexes. Nat Biotechnol. 18(1):97-100.
S. M. Hammond et al., Nat. Rev. Genet. 2, 110-119 (2001)
Higuchi, T. and Stella, V., “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.
G. Hutvágner et al., Curr. Opin. Genet. Dev. 12, 225-232 (2002).
G. Hutvágner et al., Science 297, 2056-2060 (2002).
Kelley, R. J., Brickman, M. J. and Balber, A. E. (1995) Processing and transport of a lysosomal membrane glycoprotein is developmentally regulated in African trypanosomes. Mol. Biochem. Parasitol. 74:167-178.
Lai, C. (1994). “Genetic applications of transposable elements in eukaryotes.” Genome, 37(4), 519-525.
Lewis S A, Grubman M J. (1991) VP2 is the major exposed protein on orbiviruses. Arch Virol. 121 (l-4):233-6.
Lotze, M. T., and Kost, T. A. (2002). “Viruses as gene delivery vectors: Applications to gene function, target validation, and assay development.” Cancer Gene Ther, 9(8), 692-699.
J. C. Morris, Z. Wang, M. E. Drew, K. S. Paul, and P. T. Englund. Inhibition of bloodstream form Trypanosoma brucei gene expression by RNA interference using the pZJM dual T7 vector. Mol. Biochem. Parasitol. 117 (1): 111-113, 2001.
Pearson, T. W., Beecroft, R. P., Welbum, S. C., Ruepp, S., Roditi, I., Hwa, K. Y., Englund, P. T., Wells, C. W., and Murphy, N. B. (2000). “The major cell surface glycoprotein procyclin is a receptor for induction of a novel form of cell death in African trypanosomes in vitro.” Mol Biochem Parasitol, 111(2), 333-349.
Porcel, B. M., Tran, A. -N., Tammi, M., Nyarady, Z., Rydaker, M., Urmenyi, T. P., Rondinelli, E., Pettersson, U., Anderson, B., and Aslund, L. (2000). “Gene survey of the pathogenic protozoan Trypanosoma cruzi.” Genome Research, 10, 1103-1107.
Santos, W. G. d., and Buck, G. A. (2000). “Simultaneous stable expression of neomycin phosphotransferase and green fluorescence protein genes in Trypanosoma cruzi.” J Parasitol, 86(6), 1281-1288.
P. A. Sharp et al., Genes Dev. 15, 485-490 (2001).
H. Shi, S. Wormsley, C. Tschudi, and E. Ullu. Efficient transposition of preformed synaptic Tn5 complexes in Trypanosoma brucei. Mol. Biochem. Parasitol. 121 (1):141-144, 2002.
Shimamura, M., Hager, K. M., and Hajduk, S. L. (2001). “The lysosomal targeting and intracellular metabolism of trypanosome lytic factor by Trypanosoma brucei brucei.” Molecular & Biochemical Parasitology, 115, 227-237.
Stoka, V., Turk, B., Schendel, S. L., Kim, T. -H., Cirman, T., Snipas, S. J., Ellerby, L. M., Bredesen, D., Freeze, H., Abrahamson, M., Bromme, D., Krajewski, S., Reed, J. C., Yin, X. -M., Turk, V., and Salvesen, G. S. (2001). “Lysosomal Protease Pathways to Apoptosis.” Journal of Biological Chemistry, 276(5), 3149-3157.
Tan, S. A modular polycistronic expression system for overexpressing protein complexes in Escherichia coli. Protein Expression and Purification 2001, 21: 224-294.
Teixeira, A. R. L., Arganaraz, E. R., Jr., L. H. F., Lacava, Z. G. M., and Santana, J. M. (1994). “Possible integration of Trypanosoma cruzi kDNA minicircles into the host cell genome by infection.” Mutation Research, Fundamental and Molecular Mechanisms of Mutagenesis, 305(2), 197-209.
Trudeau, C., Yuan, S., Galipeau, J., Benlimame, N., Alaoui-Jamali, M. A., and Batist, G. (2001). “A novel parasite-derived suicide gene for cancer gene therapy with specificity for lung cancer cells.” Hum Gene Ther, 12(13), 1673-1680.
Turner, C. M. R., Sternberg, J., Buchanan, N., Smith, E., Hide, G., and Tait, A. (1990). “Evidence that the mechanism of gene exchange in Trypanosoma brucei involves meiosis and syngamy.” Parasitology, 101(3), 377-386.
vanderSpek J. C., Murphy J. R. Fusion protein toxins based on diphtheria toxin: selective targeting of growth factor receptors of eukaryotic cells. Methods Enzymol. 2000;327:239-49.
Villalta, F., Smith, C. M., Ruiz-Ruano, A., and Lima, M. F. (2001). “A ligand that Trypanosoma cruzi uses to bind to mammalian cells to initiate infection.” FEBS Letters, 505(3), 383-388.
P. M. Waterhouse et al., Nature 411, 834-842 (2001).
Weber, W., and Fussenegger, M. (2002). “Artificial mammalian gene regulation networks-novel approaches for gene therapy and bioengineering.” J of Biotechnology, 98, 161-187.
Wen, L. -M., Xu, P., Benegal, G., Carvaho, M. R. C., and Butler, D. R. (2001). “Trypanosoma cruzi: Exogenously regulated gene expression.” Experimental Parasitology, 97, 196-204.
Wirtz, E., Leal, S., Ochatt, C., and Cross, G. M. (1999). “A tightly regulated inducible expression system for conditional gene knock-outs and dominant-negative genetics in Trypanosoma brucei.” Mol Biochem Parasitol, 99(1), 89-101.

Any patents, applications or publications mentioned in the specification are indicative of the levels of those skilled in the art to which the invention pertains. These patents, applications and publications are herein incorporated by reference to the same extent as if each individual publication was specifically and individually indicated to be incorporated by reference.

In view of the teaching presented herein, other modifications and variations of the present invention will readily be apparent to those of skill in the art. The discussion and description are illustrative of some embodiments of the present invention, but are not meant to be limitations on the practice thereof. It is the following claims, including all equivalents, which define the scope of the invention.

1. A therapeutic delivery system for a host comprising:
a therapeutic agent; and
a sacromastigophoric organism containing said therapeutic agent and a recombinant lytic factor.

2. The system of claim 1 wherein said therapeutic agent is selected from the group consisting of:
a gene, an artificial chromosome, magnetic species, radioactive species, vitamins, nanocrystals, drugs, and prodrugs.

3. The system of claim 2 wherein said therapeutic agent is a gene selected from the group consisting of: a native organism gene, a host gene, a pathogen gene, a polymorph of a host gene, a polymorph of a pathogen gene, a virus, and a provirus.

4. The system of claim 1 wherein the said organism is selected from the group consisting of Trypanosoma, Plasmodium, Amoeba, Giardia, Entamoeba, and Leishmania.

5. The system of claim 1 wherein said lytic factor is selected from the group consisting of: Hpr, trialysin, Bad and Bax.

6. The system of claim 5 wherein said trypanosome is Trypanosoma brucei.

7. The system of claim 1 wherein said recombinant lytic factor is upregulated by a promoter responsive to an induction species exogenous to both said organism and said host.

8. The system of claim 7 wherein said induction species is an antibiotic.

9. The system of claim 1 further comprising a gene encoding a small interfering RNA related to said therapeutic agent.

10. The system of claim 1 wherein said therapeutic agent is a diagnostic marker.

11. A therapeutic delivery system for a host comprising:
a trypanosome organism containing a recombinant lytic factor upregulated by a promoter responsive to an induction species exogenous to both said organism and said host.

12. The system of claim 11 further comprising an expression cassette having a translatable gene coding for a polypeptide.

13. The system of claim 11 wherein said trypanosome is Trypanosoma brucei.

14. The system of claim 12 wherein said gene codes green fluorescent protein.

15. The system of claim 12 wherein said expression cassette further comprises a plurality of translatable genes.

16. A process for producing a sacromastigophoric organism for delivery of a therapeutic agent comprising the steps of:
culturing sacromastigophoric organisms that have been transfected with an expression cassette induced by a first exogenous species, the cassette comprising:
a first construct having a first promoter controlling expression of a lytic protein.

17. The process of claim 16 wherein said organism is selected from the group consisting of:
Trypanosoma, Plasmodium, Amoeba, Giardia, Entamoeba, and Leishmania.

18. The process of claim 16 wherein said organism is a Trypanosoma.

19. The process of claim 18 wherein said organism is Trypanosoma brucei.

20. The process of claim 16 further comprising a second construct encoding genes comprising a second promoter, a polymerase termination sequence, and a preselected gene.

21. The process of claim 20 wherein said second construct further comprises a ribosome binding site and a poly A tail.

22. The process of claim 20 further comprising a gene conferring resistance to a second exogenous species.

23. The process of claim 16 wherein said first promoter is induced by said exogenous species.

24. The process of claim 16 wherein said first exogenous species is an antibiotic.

25. The process of claim 16 further comprising the step of packaging a non-nucleic acid therapeutic agent in said organism.

26. A process for producing a sacromastigophoric organism for delivery of a therapeutic agent comprising the steps of:
culturing trypanosome organisms that have been transfected with an expression cassette induced by a first exogenous species, the cassette comprising:
a first construct having a promoter induced by said first exogenous species controlling expression of haptoglobin related protein.

27. The process of claim 26 further comprising a second construct encoding genes comprising a second promoter, a polymerase termination sequence, and a preselected gene.

28. The process of claim 27 wherein said second construct further comprises a ribosome binding site and a poly A tail.

29. The process of claim 27 further comprising a gene conferring resistance to a second exogenous species.

30. The process of claim 26 wherein said first exogenous species is an antibiotic.

31. The process of claim 22 wherein said second exogenous species is an antibiotic effective against a wild trypanosome.

32. A method of treating or preventing a disease in a host comprising the steps of:
administering to said host a therapeutic amount of a sacromastigophoric organism that has been transfected with an expression cassette induced by an exogenous species signal, said cassette comprising a first construct having a promoter controlling expression of lytic protein;
allowing sufficient time for said organism to infect said host; and
administering said exogenous species to induce lysis of said organism.

33. The method of claim 32 wherein said organism is selected from the group consisting of:
Trypanosoma, Plasmodium, Amoeba, Giardia, Entamoeba, and Leishmania.

34. The method of claim 32 wherein said organism is Trypanosoma brucei.

35. The method of claim 32 wherein said exogenous species is an antibiotic.

36. The method of claim 32 further comprising the step of introducing into said organism a second construct encoding genes comprising:
a second promoter, a polymerase termination sequence, integrase, and a preselected gene.

37. The method of claim 36 wherein said preselected gene encodes a host gene, a pathogen gene, a polymorph of a host gene, a polymorph of a pathogen gene, a virus, and a provirus.

38. The method of claim 32 further comprising the step of packaging a non-nucleic acid therapeutic agent into said organism prior to administering said organism to said host.

39. The method of claim 38 wherein said non-nucleic acid therapeutic agent is selected from a group consisting of: magnetic species, radioactive species, vitamins, nanocrystals, drugs, and prodrugs.

40. The use of an intracellular parasite containing a recombinant exogenous species induced lytic factor to deliver a therapeutic agent to a host.

41. An organism obtainable by the process as claimed in claim 16.

42. A commercial package comprising a therapeutic agent delivery system according to claim 1 as an active ingredient with instructions for the use thereof as a therapeutic.
